(19) United States
(12) Patent Application Publication
(10) Pub. No.: US 2005/0226857 A1
(43) Pub. Date: Oct. 13, 2005

(54) T cell therapy for the treatment of cachexia and chronic diseases

(75) Inventors: Mark Bonyhadi, Issaquah, WA (US); Ronald J. Berenson, Mercer Island, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: XCYTE Therapies, Inc., Seattle, WA (US)

(21) Appl. No.: 11/091,361

(22) Filed: Mar. 28, 2005

Related U.S. Application Data
(63) Continuation-in-part of application No. 10/162,227, filed on Jun. 3, 2002.
(60) Provisional application No. 60/295,200, filed on Jun. 1, 2001.

(51) Int. Cl. A61K 45/00; C12N 5/08

(52) U.S. Cl. 424/93.7; 435/372

(57) ABSTRACT

The present invention relates to compositions and methods for the use of T cells and more particularly, activated T cells, in the treatment and/or amelioration of diseases associated with a proinflammatory state, such as cachexia, chronic diseases such as chronic renal failure, and hepatitis.

### BACKGROUND OF THE INVENTION

1. Field of the Invention

The present invention relates generally to compositions and methods for the use of T cells, particularly activated T cells, in the amelioration and/or treatment of cachexia and other disorders associated with a proinflammatory state (e.g., chronic diseases such as chronic renal failure and hepatitis).

2. Description of the Related Art

Cachexia is a syndrome that encompasses a wide range of metabolic, hormonal, and cytokine-related abnormalities that result in a wasting syndrome. While classically associated with cancer, cachexia is present in patients with a variety of chronic illnesses including HIV, cancer, chronic infections such as hepatitis and tuberculosis, chronic organ failure including renal failure, liver failure, heart failure, chronic obstructive pulmonary disease, and the like. Cachexia is characterized by anorexia, weight loss, premature satiety, asthenia, loss of lean body mass, and multiple organ dysfunction. The majority of patients with cancer whose disease progresses to metastatic disease develop cachexia during their treatment program and the cachexia contributes to their deaths. The frequency of weight loss in cancer patients ranges from 40% for patients with breast cancer, acute myelocytic leukemia, and sarcoma to more than 80% in patients with carcinoma of the pancreas and stomach. About 60% of patients with carcinomas of the lung, colon or prostate have experienced weight loss prior to beginning chemotherapy. Although the relationship between pretreatment malnutrition (weight loss) and adverse outcome is established, no consistent relationship has been demonstrated between the development of cachexia and tumor size, disease stage, and type or duration of the malignancy. Development of cachexia in cancer patients is not caused simply by increased energy expenditure by the host or by the tumor. The cancer cachexia is partially related to reduced caloric intake.

Cancer cachexia is not simply a local effect of the tumor. Alterations in protein, fat, and carbohyrate metabolism occur commonly. For example, abnormalities in carbohydrate metabolism include increased rates of total glucose turnover, increased hepatic gluconeogenesis, glucose intolerance and elevated glucose levels. Increased lipolysis, increased free fatty acid and glycerol turnover, hyperlipidemia, and reduced lipoprotein lipase activity are frequently noted. The weight loss associated with cancer cachexia is caused not only by a reduction in body fat stores but also by a reduction in total body protein mass, with extensive skeletal muscle wasting. Increased protein turnover and poorly regulated amino acid oxidation may also be important. Presence of host-derived factors produced in response to the cancer have also been implicated as causative agents of cachexia, e.g., tumor necrosis factor-alpha (TNF-alpha) or cachectin, interleukin-1 (IL-1), IL-6, gamma-interferon (IFN), and prostaglandins (PGs) (e.g; PGE₂).

Thus, the prevention and/or treatment of cachexia remain a frustrating problem. Both animal and human studies suggest that nutritional support is largely ineffective in repleting lean body mass in the cancer-bearing host. Randomized trials exploring the usefulness of total parenteral nutrition (TPN) support as an adjunct to cytotoxic antineoplastic therapy have demonstrated little improvement in treatment results. See for example Brennan, M. F., and Burt, M. E, 1981, Cancer Treatment Reports 65 (Suppl. 5): 67-68. This, along with a demonstration that TPN can stimulate tumor growth in animals suggests the routine use of TPN in cancer treatment is not justified. Kisner, D. L, 1981, Cancer Treatment Reports 65 (Suppl. 5): 1-2. Accordingly, there is a need in the art for effective treatments for cachexia.

### SUMMARY OF THE INVENTION

One aspect of the present invention provides a method for treating (e.g., ameliorating as measured by any of a varitey of indicators known in the art and described herein) a disease associated with a proinflammatory state in an individual, comprising contacting a population of cells, wherein at least a portion of the population comprises T cells, from an individual afflicted with the disease, with a surface wherein said surface has attached thereto a first agent which stimulates a TCR/CD3 complex-associated signal in the T cells and a second agent that binds the CD28 accessory molecule on the surface of the T cells, thereby activating the T cells; administering the activated T cells to the individual; thereby ameliorating the disease associated with a proinflammatory state in the individual. In one embodiment, the disease is a chronic inflammatory condition, e.g., chronic cardiac disease, chronic lung disease, chronic renal failure, hepatitis, chronic autoimmune disease, and chronic infections. In a further embodiment, the disease is cachexia. In another embodiment of the methods provided herein, the treatment and/or ameliorating comprises or otherwise results in a reduction in serum levels of one or more proinflammatory cytokines as compared to levels prior to administering the activated T cells. In an additional embodiment, the treatment leads to an increase in body weight, and/or to any one or more of an increase in energy level, an increase in ECOG performance status, and an increase in Karofsky performance status.

In a further embodiment of the methods provided herein, the first agent is an antibody or an antigen-binding fragment thereof. In certain embodiments the the antibody or antigen-binding fragment thereof is a monoclonal antibody or antigen-binding fragement thereof and in some cases, the antibody is an anti-CD3 antibody. In yet a further embodiment, the second agent is an antibody or an antigen-binding fragment thereof and in certain embodiments, the antibody or antigen-binding fragment thereof is a monoclonal antibody or antigen-binding fragement thereof. In one embodiment, the second agent antibody is an anti-CD28 antibody. In a further embodiment, the first and the second agents are both antibodies or antigen-binding fragments thereof. In this regard, the first agent is may be an anti-CD3 antibody or antigen-binding fragments thereof and the second agent may be an anti-CD28 antibody or antigen-binding fragments thereof. In a further embodiment, the second agent is a natural ligand of CD28, such as B7-1.

In another embodiment, the surface may be a solid surface, a cell surface, or a paramagnetic bead. In this regard, in certain embodiments, the first and second agents are attached to the surface by a variety of means, e.g., the first and said second agent are covalently attached, noncovalently to the surface. In certain embodiments, the first and second agent are indirectly attached to the surface.

Another aspect of the present invention, provides a method for treating a disease associated with a proinflammatory state, comprising administering activated T cells to an individual afflicted with the disease; thereby treating the disease associated with a proinflammatory state. In one embodiment, the disease is a chronic inflammatory condition such as, but not limited to, chronic cardiac disease, chronic lung disease, chronic renal failure, hepatitis, chronic autoimmune disease, and chronic infections. In one embodiment, the disease is cachexia. In a further embodiment of the method, the treatment results in a reduction in serum levels of one or more proinflammatory cytokines as compared to levels prior to administering the activated T cells. In yet another embodiment, the treatment leads to an increase in body weight, and/or to any one or more of an increase in energy level, an increase in ECOG performance status, and an increase in Karofsky performance status.

### BRIEF DESCRIPTION OF THE DRAWINGS

FIG. 1: CT scan from a patient before and after treatment with activated T cells (XCELLERATE™) treatment.

FIG. 2: CT scan from a patient before and 3 and 4 months after XCELLERATE™ treatment.

### DETAILED DESCRIPTION OF THE INVENTION

Prior to setting forth the invention, it may be helpful to an understanding thereof to set forth definitions of certain terms that will be used hereinafter.

The term “stimulation”, as used herein, refers to a primary response induced by ligation of a cell surface moiety. For example, in the context of receptors, such stimulation entails the ligation of a receptor and a subsequent signal transduction event. With respect to stimulation of a T cell, such stimulation refers to the ligation of a T cell surface moiety that in one embodiment subsequently induces a signal transduction event, such as binding the TCR/CD3 complex. Further, the stimulation event may activate a cell and up or downregulate expression or secretion of a molecule, such as downregulation of Tumor Growth Factor beta (TGF-β). Thus, ligation of cell surface moieties, even in the absence of a direct signal transduction event, may result in the reorganization of cytoskeletal structures, or in the coalescing of cell surface moieties, each of which could serve to enhance, modify, or alter subsequent cell responses.

The term “activation”, as used herein, refers to the state of a cell following sufficient cell surface moiety ligation to induce a measurable biochemical or morphological, phenotypic, and/or functional change. Within the context of T cells, such activation may be the state of a T cell that has been sufficiently stimulated to induce cellular proliferation. Activation of a T cell may also induce cytokine production and/or secretion, and performance of regulatory or cytolytic effector functions. Within the context of other cells, this term infers either up or down regulation of a particular physico-chemical process.

The term “target cell”, as used herein, refers to any cell that is intended to be stimulated by cell surface moiety ligation.

An “antibody”, as used herein, includes both polyclonal and monoclonal antibodies (mAb); primatized (e.g., humanized); murine; mouse-human; mouse-primate; and chimeric; and may be an intact molecule, a fragment thereof (such as scFv, Fv, Fd, Fab, Fab′ and F(ab)′₂ fragments), or multimers or aggregates of intact molecules and/or fragments; and may occur in nature or be produced, e.g., by immunization, synthesis or genetic engineering; an “antibody fragment,” as used herein, refers to fragments, derived from or related to an antibody, which bind antigen and which in some embodiments may be derivatized to exhibit structural features that facilitate clearance and uptake, e.g., by the incorporation of galactose residues. This includes, e.g., F(ab), F(ab)′₂, scFv, light chain variable region (V_{L}), heavy chain variable region (V_{H}), and combinations thereof.

The term “protein”, as used herein, includes proteins, glycoproteins and other cell-derived modified proteins, polypeptides and peptides; and may be an intact molecule, a fragment thereof, or multimers or aggregates of intact molecules and/or fragments; and may occur in nature or be produced, e.g., by synthesis (including chemical and/or enzymatic) or genetic engineering.

The term “agent”, “ligand”, or “agent that binds a cell surface moiety”, as used herein, refers to a molecule that binds to a defined population of cells. The agent may bind any cell surface moiety, such as a receptor, an antigenic determinant, or other binding site present on the target cell population. The agent may be a protein, peptide, antibody and antibody fragments thereof, fusion proteins, synthetic molecule, an organic molecule (e.g., a small molecule), or the like. Within the specification and in the context of T cell stimulation, antibodies are used as a prototypical example of such an agent.

The term “cell surface moiety” as used herein may refer to a cell surface receptor, an antigenic determinant, or any other binding site present on a target cell population.

The terms “agent that binds a cell surface moiety” and “cell surface moiety”, as used herein, should be viewed as a complementary/anti-complementary set of molecules that demonstrate specific binding, generally of relatively high affinity (an affinity constant, Kₐ, of about 10⁶ M⁻¹).

A “co-stimulatory signal”, as used herein, refers to a signal, which in combination with a primary signal, such as TCR/CD3 ligation, leads to T cell proliferation and/or activation.

“Separation”, as used herein, includes any means of substantially purifying one component from another (e.g., by filtration, affinity, buoyant density, or magnetic attraction).

A “surface”, as used herein, refers to any surface capable of having an agent attached thereto and includes, without limitation, metals, glass, plastics, co-polymers, colloids, lipids, cell surfaces, and the like. Essentially any surface that is capable of retaining an agent bound or attached thereto. A prototypical example of a surface used herein, is a particle such as a bead.

“Ameliorate” as used herein, is defined as: to make better; improve (The American Heritage College Dictionary, 3^{rd} Edition, Houghton Mifflin Company, 2000).

“Particles” as used herein, may include a colloidal particle, a microsphere, nanoparticle, a bead, or the like. In the various embodiments, commercially available surfaces, such as beads or other particles, are useful (e.g., Miltenyi Particles, Miltenyi Biotec, Germany; Sepharose beads, Pharmacia Fine Chemicals, Sweden; DYNABEADS™, Dynal Inc., New York; PURABEADS™, Prometic Biosciences, magnetic beads from Immunicon, Huntingdon Valley, Pa., microspheres from Bangs Laboratories, Inc., Fishers, Ind.).

“Paramagnetic particles” as used herein, refer to particles, as defined above, that localize in response to a magnetic field.

“Antigen” as used herein, refers to any molecule 1) capable of being specifically recognized, either in its entirety or fragments thereof, and bound by the “idotypic” portion (antigen-binding region) of a mAb or its derviative; 2) containing peptide sequences which can be bound by MHC and then, in the context of MHC presentation, can specifically engage its cognate T cell antigen receptor.

The term “animal” or “mammal” as used herein, encompasses all mammals, including humans. Preferably, the animal of the present invention is a human subject.

The term “exposing” as used herein, refers to bringing into the state or condition of immediate proximity or direct contact.

The term “proliferation” as used herein, means to grow or multiply by producing new cells.

A “wound site” as used herein, is defined as any location in the host that arises from tissue injury, from tissue damage either induced by, or resulting from, surgical procedures, infection, traumatic injury, or a disease state including, but not limited to, infarcted myocardium, ischemic myocardium, eroded bone, degenerated cartalagenous tissue, degenerated nerve tissue, burns, or transplant sites.

The term “neurotrophic factor”, as used herein, refers to compounds which are capable of stimulating growth or proliferation of nervous tissue.

With respect to wound healing, an improved clinical outcome can refer to a more rapid rate of wound closure, less wound contraction and/or less scarring.

With respect to neovascularization to bypass occluded blood vessels, a “therapeutically effective amount” is a quantity which results in the formation of new blood vessels which can transport at least some of the blood which normally would pass through the blocked vessel.

T Cell Compositions

T cells are unique in their biology and function. They express on their surface and secrete an array of important molecules capable of interacting with other cells/tissues in specific manners which can facilitate or regulate the differentiation/de-differentiation/maturation/tissue organization and repair activity of those cells or tissues. In particular, T cells in various states of activation (and thus expressing differing panels of surface or secreted molecules) can lead to tissue development, differentiation or reorganization and repair which can ameliorate a variety of medical conditions.

T cells, particularly activated T cells, possess many of the potential molecules involved in the complex process of the repair and regeneration of mammalian tissues, and for amelioration of disorders such as cachexia and other diseases associated with a proinflammatory state. Accordingly, T cells fill a need in the art of providing a complex and regulated array of molecules necessary to provide regulation of aberrant cytokine cascades present in disorders such as cachexia, chronic renal failure, and hepatitis, and to provide tissue growth and/or remodeling via regulation/control of other cell types involved in this process.

Generally, the activated T cells of the present invention are generated by cell surface moiety ligation that induces activation. The activated T cells are generated by activating a population of T cells and stimulating an accessory molecule on the surface of the T cells with a ligand which binds the accessory molecule, as described for example, in U.S. patent application Ser. Nos. 10/762,210; 10/350,305; 10/187,467; 10/133,236; 08/253,694; 08/435,816; 08/592,711; 09/183,055; 09/350,202; and 09/252,150; and U.S. Pat. Nos. 6,352,694; 5,858,358 and 5,883,223; all of which are hereby incorporated by reference in their entirety.

T cells can be obtained from a number of sources, including peripheral blood mononuclear cells, bone marrow, thymus, tissue biopsy, tumor, lymph node tissue, gut associated lymphoid tissue, mucosa associated lymphoid tissue, spleen tissue, or any other lymphoid tissue, and tumors. T cells can be obtained from T cell lines and from autologous or allogeneic sources. T cells may also be obtained from a xenogeneic source, for example, from mouse, rat, non-human primate, and pig.

Preferably, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis or leukapheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, Baxter) according to the manufacturer's instructions. After washing, the cells may be resuspended in a variety of biocompatible buffers, such as, for example, Ca⁺⁺/Mg⁺⁺ free PBS. Alternatively, the undesirable components of the apheresis sample may be removed and the cells directly resuspended in culture media.

In another embodiment, T cells are isolated from peripheral blood lymphocytes by lysing the red blood cells, isolating and reserving the monocytes as described previously, or for example, by centrifugation through a PERCOLL™ gradient. A specific subpopulation of T cells, such as CD28⁺, CD4⁺, CD8⁺, CD45RA⁺, and CD45RO⁺T cells, can be further isolated by positive or negative selection techniques. For example, CD3⁺, CD28⁺ T cells can be positively selected using CD3/CD28 conjugated magnetic beads (e.g., DYNABEADS® M-450 CD3/CD28 T Cell Expander). In one aspect of the present invention, enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8.

Accordingly, in one embodiment, the invention uses paramagnetic particles of a size sufficient to be engulfed by phagocytotic monocytes, that are subsequently removed through magnetic separation. In certain embodiments, the paramagnetic particles are commercially available beads, for example, those produced by Dynal AS under the trade name Dynabeads™. Exemplary Dynabeads™ in this regard are M-280, M-450, and M-500. In one aspect, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be expanded. In certain embodiments, the irrelevant beads include beads coated with sheep anti-mouse antibodies, goat anti-mouse antibodies, and human serum albumin.

Another method to prepare the T cells for stimulation is to freeze the cells after the washing step, which does not require the monocyte-removal step. Wishing not to be bound by theory, the freeze and subsequent thaw step provides a more uniform product by removing granulocytes and, to some extent, monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

The activated T cells of the present invention are generated by cell surface moiety ligation that induces activation. The activated T cells are generated by activating a population of T cells and stimulating an accessory molecule on the surface of the T cells with a ligand which binds the accessory molecule, as described for example, in U.S. patent application Ser. Nos. 10/762,210; 10/350,305; 10/187,467; 10/133,236; 08/253,694; 08/435,816; 08/592,711; 09/183,055; 09/350,202; and 09/252,150; and U.S. Pat. Nos. 6,352,694; 5,858,358 and 5,883,223, all of which are hereby incorporated by reference in their entirety.

Generally, T cell activation may be accomplished by cell surface moiety ligation, such as stimulating the T cell receptor (TCR)/CD3 complex or the CD2 surface protein with an agent as described herein. Exemplary agents include, but are not limited to, antibodies. A number of anti-human CD3 monoclonal antibodies are commercially available, exemplary are, clone BC3 (XR-CD3; Fred Hutchinson Cancer Research Center, Seattle, Wash.), OKT3, prepared from hybridoma cells obtained from the American Type Culture Collection, and monoclonal antibody G19-4. Similarly, stimulatory forms of anti-CD2 antibodies are known and available. Stimulation through CD2 with anti-CD2 antibodies is typically accomplished using a combination of at least two different anti-CD2 antibodies. Stimulatory combinations of anti-CD2 antibodies that have been described include the following: the T11.3 antibody in combination with the T11.1 or T11.2 antibody (Meuer et al., Cell 36:897-906, 1984), and the 9.6 antibody (which recognizes the same epitope as T11.1) in combination with the 9-1 antibody (Yang et al., J. Immunol. 137:1097-1100, 1986). Other antibodies that bind to the same epitopes as any of the above described antibodies can also be used. Additional antibodies, or combinations of antibodies, can be prepared and identified by standard techniques. Stimulation may also be achieved through contact with antigen, peptide, protein, peptide-MHC tetramers (see Altman, et al. Science Oct. 4, 1996; 274(5284):94-6), superantigens (e.g., Staphylococcus enterotoxin A (SEA), Staphylococcus enterotoxin B (SEB), Toxic Shock Syndrome Toxin 1 (TSST-1)), endotoxin, or through a variety of mitogens, including but not limited to, phytohemagglutinin (PHA), phorbol myristate acetate (PMA) and ionomycin, lipopolysaccharide (LPS), T cell mitogen, and IL-2.

To further activate a population of T cells, a co-stimulatory or accessory molecule on the surface of the T cells, such as CD28, is stimulated with an agent (e.g., an antibody or a natural ligand) that binds the accessory molecule. Accordingly, one of ordinary skill in the art will recognize that any agent, including an anti-CD28 antibody or fragment thereof capable of cross-linking the CD28 molecule, or a natural ligand for CD28 can be used to stimulate T cells. Exemplary anti-CD28 antibodies or fragments thereof useful in the context of the present invention include monoclonal antibody 9.3 (IgG2ₐ) (Bristol-Myers Squibb, Princeton, N.J.), monoclonal antibody KOLT-2 (IgG1), 15E8 (IgG1), 248.23.2 (IgM), clone B-T3 (XR-CD28; Diaclone, Besançon, France) and EX5.3D10 (IgG2ₐ) (ATCC HB11373). Exemplary natural ligands include the B7 family of proteins, such as B7-1 (CD80) and B7-2 (CD86) (Freedman et al., J. Immunol. 137:3260-3267, 1987; Freeman et al, J. Immunol. 143:2714-2722, 1989; Freeman et al., J. Exp. Med. 174:625-631, 1991; Freeman et al., Science 262:909-911,.1993; Azuma et al., Nature 366:76-79, 1993; Freeman et al., J. Exp. Med. 178:2185-2192, 1993).

In addition, binding homologues of a natural ligand, whether native or synthesized by chemical or recombinant techniques, can also be used in accordance with the present invention. Other agents may include natural and synthetic ligands. Agents may include, but are not limited to, other antibodies or fragments thereof, a peptide, polypeptide, growth factor, cytokine, chemokine, glycopeptide, soluble receptor, steroid, hormone, mitogen, such as PHA, or other superantigens.

The primary stimulatory signal and the co-stimulatory signal for the T-cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing FC receptors or an antibody or other binding agent which will bind to the agents. In a preferred embodiment, the two agents are immobilized on beads, either on the same bead, i.e., “cis,” or to separate beads, i.e., “trans.” By way of example, the agent providing the primary activation signal is an anti-CD3 antibody and the agent providing the co-stimulatory signal is an anti-CD28 antibody; and both agents are co-immobilized to the same bead in equivalent molecular amounts. In one embodiment, a 1:1 ratio of each antibody bound to the beads for CD4⁺ T-cell expansion and T-cell growth is used. In certain aspects of the present invention, a ratio of anti CD3:CD28 antibodies bound to the beads is used such that an increase in T cell expansion is observed as compared to the expansion observed using a ratio of 1:1. In one particular embodiment an increase of from about 0.5 to about 3 fold is observed as compared to the expansion observed using a ratio of 1:1. In one embodiment, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e. the ratio of CD3:CD28 is less than one. In certain embodiments of the invention, the ratio of anti CD28 antibody to anti CD3 antibody bound to the beads is greater than 2:1. In one particular embodiment, a 1:200 CD3:CD28 ratio of antibody bound to beads is used. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one preferred embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells from 1:500 to 500:1 and any integer values in between may be used to stimulate T-cells or other target cells. As those of ordinary skill in the art can readily appreciate, the ratio of particle to cells may dependant on particle size relative to the target cell. For example, small sized beads could only bind a few cells, while larger beads could bind many. In certain embodiments the ratio of cells to particles ranges from 1:100 to 100:1 and any integer values in between and in further embodiments the ratio comprises 1:9 to 9:1 and any integer values in between, can also be used to stimulate T-cells. The ratio of anti-CD3- and anti-CD28-coupled beads particles to T-cells that result in T-cell stimulation can vary as noted above, however in certain embodiments, the ratio of anti-CD3 and anti-CD28 coupled beads to cells includes 1:100, 1:50, 1:40, 1:30, 1:20, 1:15, 1:10, 1:5, 1:4, 1:3, 1:2, 1:1, 2:1, 3:1, 4:1 to 6:1, with one particular ratio being 3:1 beads/particles per T-cell. In one embodiment, a ratio of particles to cells of 1:1 or less is used. In further embodiments, the ratio of particles to cells can be varied depending on the day of stimulation. For example, in one embodiment, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In another embodiment, particles are added on a daily or every other day basis to a final ratio of 1:1 on the first day, and 1:5 on the third and fifth days of stimulation. In another embodiment, the ratio of particles to cells is 2:1 on the first day of stimulation and adjusted to 1:10 on the third and fifth days of stimulation. In another embodiment, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type.

Using certain methodologies it may be advantageous to maintain long-term stimulation of a population of T-cells following the initial activation and stimulation, by separating the T-cells from the stimulus after a period of about 12 to about 14 days. The rate of T-cell proliferation is monitored periodically (e.g., daily) by, for example, examining the size or measuring the volume of the T-cells, such as with a Coulter Counter. In this regard, a resting T-cell has a mean diameter of about 6.8 microns, and upon initial activation and stimulation, in the presence of the stimulating ligand, the T-cell mean diameter will increase to over 12 microns by day 4 and begin to decrease by about day 6. When the mean T-cell diameter decreases to approximately 8 microns, the T-cells may be reactivated and re-stimulated to induce further proliferation of the T-cells. Alternatively, the rate of T-cell proliferation and time for T-cell re-stimulation can be monitored by assaying for the presence of cell surface molecules, such as , CD154, CD54, CD25, CD137, CD134, B7-1, B7-2, which are induced on activated T-cells.

For inducing long-term stimulation of a population of CD4⁺ and/or CD8⁺ T-cells, it may be necessary to reactivate and re-stimulate the T-cells with a stimulatory agent such as an anti-CD3 antibody and an anti-CD28 antibody (such as B-T3, XR-CD28 (Diaclone, Besançon, France) or monoclonal antibody ES5.2D8 several times to produce a population of CD4⁺ or CD8⁺ cells increased in number from about 10 to about 1,000-fold the original T-cell population. For example, in one embodiment of the present invention, T-cells are stimulated as described herein for 2-3 times. In further embodiments, T-cells are stimulated as described herein for 4 or 5 times.

In another embodiment, the time of exposure to stimulatory agents such as anti-CD3/anti-CD28 (i.e., CD3×CD28)-coated beads may be modified or tailored to obtain a desired T-cell phenotype. One may desire a greater population of helper T-cells (T_{H}), typically CD4⁺ as opposed to CD8⁺ cytotoxic or suppressor T-cells (T_{C}), because an expansion of T_{H} cells could induce desired tissue repair and/or regeneration. CD4⁺ T-cells, express important immune-regulatory molecules, such as GM-CSF, CD40L, and IL-2, for example. Where CD4-mediated help is preferred, a method, such as that described herein, which preserves or enhances the CD4:CD8 ratio could be of significant benefit. In one aspect of the present invention, it may be beneficial to increase the number of infused cells expressing GM-CSF, or IL-2, all of which are expressed predominantly by CD4⁺ T-cells. Alternatively, in situations where CD4-help is needed less and increased numbers of CD8⁺ T-cells are desirous, the T cell activation approaches described herein can also be utilized, by for example, pre-selecting for CD8⁺ cells prior to stimulation and/or culture. Such situations may exist where increased levels of IFN-γ is preferred. Further, in other applications, it may be desirable to utilize a population of T_{H}1-type cells versus T_{H}2-type cells (or vice versa), or supernatants therefrom. To effectuate isolation of different T-cell populations, times of cell surface moiety ligation that induces activation may be varied or pulsed. For example expansion times may be varied to obtain the specific phenotype of interest and/or different types of stimulatory agents may be used (e.g., antibodies or fragments thereof, a peptide, polypeptide, MHC/peptide tetramer, growth factor, cytokine, chemokine, glycopeptide, soluble receptor, steroid, hormone, mitogen, such as PHA, or other superantigens). The expression of a variety of phenotypic markers change over time; therefore, a particular time point or stimulatory agent may be chosen to obtain a specific population of T-cells. Accordingly, depending on the cell type to be stimulated, the stimulation and/or expansion time may be four weeks or less, 2 weeks or less, 10 days or less, or 8 days or less (four weeks or less includes all time ranges from 4 weeks down to 1 day (24 hours)). In some embodiments, stimulation and expansion may be carried out for 6 days or less, 4 days or less, 2 days or less, and in other embodiments for as little as 24 or less hours, and preferably 4-6 hours or less (these ranges include any integer values in between). When stimulation of T-cells is carried out for shorter periods of time, the population of T-cells may not increase in number as dramatically, but the population will provide more robust and healthy activated T-cells that can continue to proliferate in vivo and more closely resemble the natural effector T-cell pool.

T-cells that have been exposed to varied stimulation times and agents may exhibit different characteristics. For example, typical blood or apheresed peripheral blood mononuclear cell products have a helper T-cell population (T_{H}, CD4⁺) that is greater than the cytotoxic or suppressor T-cell population (T_{C}, CD8⁺). Ex vivo expansion of T-cells by stimulating CD3 and CD28 receptors produces a population of T-cells that prior to about days 8-9 consists predominately of T_{H} cells, while after about days 8-9, the population of T-cells comprises an increasingly greater population of T_{C} cells. Accordingly, depending on the purpose of treatment, infusing a subject with or applying a T-cell population comprising predominately of T_{H} cells may be advantageous.

Further, in addition to CD4 and CD8 markers, other phenotypic markers vary significantly, but in large part, reproducibly during the course of the cell expansion process. Thus, such reproducibility enables the ability to tailor an activated T-cell product for specific purposes (for example, for bone regeneration as opposed to angiogenesis).

In one such example, among the important phenotypic markers that reproducibly vary with time are the high affinity IL-2 receptor (CD25), CD40 ligand (CD154), and CD45RO (a molecule that by preferential association with the TCR may increase the sensitivity of the TCR to antigen binding). As one of ordinary skill in the art readily appreciates, such molecules are important for a variety of reasons. For example, CD25 constitutes an important part of the autocrine loop that allows rapid T-cell division. CD154 has been shown to play a key role in stimulating maturation of the antigen-presenting dendritic cells; activating B-cells for antibody production; regulating T_{H} cell proliferation; enhancing T_{C} cell differentiation; regulating cytokine secretion of both T_{H} cells and antigen-presenting cells; and stimulating expression of co-stimulatory ligands, including CD80, CD86, and CD154.

Production of cytokines, cell surface receptors, and other factors important in the treatment of cachexia, chronic diseases such as chronic renal failure, chronic hepatitis and tissue repair and regeneration of the present invention, increases, often starting very early, in the ex vivo expansion process. Accordingly, because cytokines and other factors are known to be important for mediating T-cell activation and function as well as modulation of cell differentiation, such factors are likely critical in the development of a therapeutic T-cell product. Molecules important in this regard, include, but are not limited to, IL-2, IL-4, TNF-α, and IFN-γ, transforming growth factor (TGF) TGF-β, neuroleukin (phosphoglucose isomerase), nerve growth factor, NF-kappaB transcription factors, and CD40. Thus, by obtaining a population of T-cells during the first few days of expansion and infusing these cells into a subject, or application of these cells or supernatants therefrom directly on an injury site, a therapeutic benefit may occur in which additional activation and expansion of T-cells in vivo occurs, and/or tissue repair and regeneration occurs.

In addition to the cytokines and the markers discussed previously, expression of adhesion molecules known to be important for mediation of T-cell activation and immune-mediated modulation of target cells also change dramatically but reproducibly over the course of the ex vivo expansion process. For example, CD62L is important for homing of T-cells to lymphoid tissues and trafficking T-cells to sites of inflammation. Because down-regulation of CD62L occurs early following activation, the T-cells could be expanded for shorter periods of time. Conversely, longer periods of time in culture would generate a T-cell population with higher levels of CD62L and thus a higher ability to target the activated T-cells to these sites under other preferred conditions. Another example of a polypeptide whose expression varies over time is CD49d, an adhesion molecule that is involved in trafficking lymphocytes from blood to tissues spaces at sites of inflammation. Binding of the CD49d ligand to CD49d also allows the T-cell to receive co-stimulatory signals for activation and proliferation through binding by VCAM-1 or fibronectin ligands. The expression of the adhesion molecule CD54, involved in T-cell-APC and T-cell-T-cell interactions as well as homing to sites of inflammation, also changes over the course of expansion. Accordingly, T-cells could be stimulated for selected periods of time that coincide with the marker profile of interest and subsequently collected and infused. Compositions comprising supernatants from activated T cells could also be infused. Activated T cells, or supernatants therefrom, could also be applied directly to an injury site. Thus, T-cell populations could be tailored to express the markers believed to provide the most therapeutic benefit for the indication to be treated.

In the various embodiments, one of ordinary skill in the art understands removal of the stimulation signal from the cells is dependent upon the type of surface used. For example, if paramagnetic beads are used, then magnetic separation is the feasible option. Separation techniques are described in detail by paramagnetic bead manufacturers' instructions (for example, DYNAL Inc., Oslo, Norway). Furthermore, filtration may be used if the surface is a bead large enough to be separated from the cells. In addition, a variety of transfusion filters are commercially available, including 20 micron and 80 micron transfusion filters (Baxter). Accordingly, so long as the beads are larger than the mesh size of the filter, such filtration is highly efficient. In a related embodiment, the beads may pass through the filter, but cells may remain, thus allowing separation.

Although the antibodies used in the methods described herein can be readily obtained from public sources, such as the ATCC, antibodies to T-cell accessory molecules and the CD3 complex can be produced by standard techniques. Methodologies for generating antibodies for use in the methods of the invention are well-known in the art.

In one aspect of the present invention, the T cells may be genetically modified using any number of methods known in the art. The T cells may be transfected using numerous RNA or DNA expression vectors known to those of ordinary skill in the art. Genetic modification may comprise RNA or DNA transfection using any number of techniques known in the art, for example electroporation (using e.g., the Gene Pulser II, BioRad, Richmond, Calif.), various cationic lipids, (LIPOFECTAMINE™, Life Technologies, Carlsbad, Calif.), or other techniques such as calcium phosphate transfection as described in Current Protocols in Molecular Biology, John Wiley & Sons, New York. N.Y. For example, 5-50 μg of RNA or DNA in 500 μl of Opti-MEM can be mixed with a cationic lipid at a concentration of 10 to 100 μg, and incubated at room temperature for 20 to 30 minutes. Other suitable lipids include LIPOFECTIN™, LIPOFECTAMINE™. The resulting nucleic acid-lipid complex is then added to 1-3×10⁶ cells, preferably 2×10⁶, antigen-presenting cells in a total volume of approximately 2 ml (e.g., in Opti-MEM), and incubated at 37° C. for 2 to 4 hours. The T cells may also be transduced using viral transduction methodologies as described below The T cells may alternatively be genetically modified using retroviral transduction technologies. In one aspect of the invention, the retroviral vector may be an amphotropic retroviral vector, preferably a vector characterized in that it has a long terminal repeat sequence (LTR), e.g., a retroviral vector derived from the Moloney murine leukemia virus (MoMLV), myeloproliferative sarcoma virus (MPSV), murine embryonic stem cell virus (MESV). murine stem cell virus (MSCV), spleen focus forming virus(SFFV), or adeno-associated virus (AAV). Most retroviral vectors are derived from murine retroviruses. Retroviruses adaptable for use in accordance with the present invention can, however, be derived from any avian or mammalian cell source. These retroviruses are preferably amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Uses for the T Cell Compositions in Tissue Repair and Regeneration

The activated T cells of the present invention can be used in the treatment of diseases associated with a proinflammatory state, such as cachexia, chronic diseases such as chronic renal failure, chronic cardiac disease, chronic autoimmune disease, and hepatitis. Further, the activated T cells of the present invention can be universally applied to damaged tissue or a tissue site in need of treatment that may involve many different cells, tissues and organs. The activated T cells are “targeted” to the sites of damaged tissue. The invention is applicable to the repair of a wide variety of damaged tissues in human medicine. These include, but are not limited to the repair and/or regeneration of eroded bone, degenerated cartilagenous tissue, ischemic myocardium, damaged endothelial cells, degenerated or otherwise damaged nerve, burn sites, post-surgical sites, and organ/tissue transplant sites.

For example, using the activated T cells, or supernatants therefrom, of the present invention, cytokine growth factors and/or other molecules produced by said cells or present in the supernatant therefrom, will influence other cells at the tissue site, through binding of cell surface signaling receptors, thereby stimulating and amplifying the cascade of physiological events normally associated with the process of wound healing, tissue repair, or remodeling. For example, the rate of wound healing would increase, leading to a more rapid re-epithelialization and tissue repair. The end result is the augmentation of tissue repair and regeneration. The cells and/or supernatants of the present invention can be used to recruit other cells, e.g. mesenchymal stem cells, bone marrow-derived angioblasts, neural stem cells, or any manner of precursor cells involved in tissue repair, including but not limited to, monocytes, to the site of injury or site in need of tissue regeneration. The activated T cells or supernatants therefrom of the present invention may be administered either in vitro or in vivo depending on the desired outcome.

The activated T cells or supernatants therefrom, of the present invention are also useful when the goal is to block a disease process, thereby allowing natural tissue healing to take place, or when the goal is to replace a genetically defective protein function.

Damaged tissue may arise from tissue injury, from tissue damage either induced by, or resulting from, surgical procedures, infection, traumatic injury, or a disease state including, but not limited to, infarcted myocardium, ischemic myocardium, eroded bone, degenerated cartalagenous tissue, degenerated nerve tissue, burns, or transplant sites. The activated T cells, or supernatants therefrom, of the present invention can be transferred to the patient using various techniques. For example, compositions comprising cells and/or supernatant therefrom can be transferred directly to the site of the wound by a physician, either as a therapeutic implant, an injection or via topical application of a suitable formulation. In addition, activated T cells or supernatants therefrom can be topically administered, or placed surgically in a normal tissue site in order to treat distal diseased tissue.

The process of wound healing is a coordinated sequence of events which includes hemorrhage, clot formation, dissolution.of the clot with concurrent removal of damaged tissue, and deposition of granulation tissue as initial repair material. The granulation tissue is a mixture of fibroblasts and capillary blood vessels. The wound healing process involves diverse cell populations including endothelial cells, stem cells, macrophages and fibroblasts. The regulatory factors involved in wound repair are known to include systemic hormones, cytokines, growth factors, extracellular matrix proteins and other proteins that regulate growth and differentiation.

The Use of T Cell Compositions in Bone Repair and Regeneration

Bone has a substantial capacity to regenerate following fracture. The complex but ordered fracture repair sequence includes hemostasis, clot dissolution, granulation tissue ingrowth, formation of a callus, and remodeling of the callus to an optimized structure (A. W. Ham. J. Bone Joint Surg. 12: 827-844, 1930). Cells participating in this process include platelets, inflammatory cells, fibroblasts, endothelial cells, pericytes, osteoclasts, and osteogenic progenitors.

Techniques designed to stimulate bone repair would be a valuable tool in treating bone fractures. A significant portion of fractured bones are still treated by casting, allowing natural mechanisms to effect wound repair. Although there have been advances in fracture treatment in recent years, including improved devices, the development of new processes to stimulate, or complement, the wound repair mechanisms would represent significant progress in this area.

The activated T cells and supernatants therefrom of the present invention may be used to promote fracture repair. Other aspects of this technology include the use of cell or supernatant transfer to treat patents with “weak bones”, such as in diseases like osteoporosis; to improve poor healing which may arise for unknown reasons, e.g., fibrous non-union; to promote implant integration and the function of artificial joints; to stimulate healing of other skeletal tissues such as Achilles tendon; and as an adjuvant to repair large defects.

Transforming growth factors (TGFs), have also been shown to have a central role in regulating tissue healing by affecting cell proliferation, gene expression, and matrix protein synthesis (Roberts & Sporn, 1989, M. B. Sporn and A. B. Roberts, Eds., Springer-Verlag, Heidelberg, 95 (Part 1)). For example, TGF-β1 and TGF-β2 can initiate both chondrogenesis and osteogenesis (Joyce et al. J. Cell Biol. 110:195-2007, 1990; Izumi et al. J. Bone Min. Res. 7:115-11, 1992; Jingushi et al. J. Orthop. Res. 8:364-371, 1992). Thus, activated T cells of the present invention, which produce this factor, can be used in the practice of the invention to influence new bone formation following fracture.

In an embodiment of the invention the activated T cells of the present invention are surgically implanted into the site of the bone fracture. Such surgical procedures may include direct injection of an activated T cell preparation into the fracture site, the surgical repair of a complex fracture, or arthroscopic surgery. In instances where the activated T cells or supernatants therefrom are being used to repair fractured bone, the mammalian repair cells will naturally migrate and proliferate at the site of bone damage.

The present invention may also be used to stimulate the growth or regeneration of soft tissues such as ligament, tendon, cartilage and skin. Skeletal connective tissue damage due to traumatic injury may be treated using the activated T cells or supernatants therefrom. Various factors produced by activated T cells can promote soft tissue repair. These include, but are not limited to, members of the TGF-β superfamily (e.g., TGF-β itself), which stimulates expression of genes coding for extracellular matrix proteins, and other cytokines such as EGF and PDGF. Examples of other factors produced by activated T cells that may be important in this process include (a) interleukins, chemokines, interferons, colony stimulating factors; (b) the family of cell adhesion molecules; (c) nuclear trans acting proteins such as transcription factors.

The activated T cells or supernatants therefrom of the present invention may be placed in the host mammal in the area of the connective tissue wound. The activated T cells or supernatants therefrom may be injected directly into the area of connective tissue injury. Alternatively, surgical techniques, such as arthroscopic surgery, may be used to deliver the cells or supernatant to the area of the connective tissue wound.

In one aspect of the present invention, the activated T cells, or supernatants therefrom, may be used to stimulate bone regeneration in in vitro cultures of bone cells, or precursors thereof. Co-culture with activated T cells or supernatant therefrom could lead to maturation, differentiation, improved function, and/or enhanced engraftment potential of the bone cells. Further, co-culture with activated T cells or supernatant therefrom of the present invention with various cell types in vitro could lead to alteration of function of the cells of non T cell lineage. The cells altered as a result of co-culture could then be administered to a mammal for use in tissue repair and/or regeneration.

The activated T cells and/or supernatants therefrom can also be used for the repair of bone metastases. There are several pathological conditions that involve irregularities in calcium and phosphate metabolism. Such conditions comprise bone related diseases including Paget's disease and osteoporosis, as well as osteolysis in bone metastases. Bone metastases present a major problem in many frequently occurring malignancies. Hypercalcemia, resulting from bone resorption, is a common and very important complication of malignancy, causing distressful symptoms, such as severe pain and spontaneous fractures, and may lead to a metabolic coma and death. Moreover, neoplastic cell-induced osteolysis may determine the localization and growth enhancement of bone tumors. (See, G. R. Mundy, Bone, 8, supp. 1, S9-5 16 (1987); and Calcium in Biological Systems, R. P. Rubin, G. B. Weiss, and J. W. Putney, Jr. eds. Plenum Press, N.Y. (1985). Other pathological conditions cause or result from deposition of calcium and phosphate anomalously in the body, such as rheumatoid arthritis and osteoarthritis. The activated T cell compositions or supernatants therefrom of the present invention can be used in the therapy of such disorders in conjunction with compounds known to facilitate the desired activity, such as the use of osteoprotegrin or bisphosphonates. Bisphosphonates are a class of drugs that have been developed for use in various metabolic diseases of bone, the target being excessive bone resorption and inappropriate calcification and ossification. (M. D. Francis and R. R. Martodam, “The Role of Phosphonates in Living Systems” R. L. Hilderbrand, ed., CRC Press, Boca Raton, Fla., 1983, pp. 55-96; and H. Fleisch, Bone, 1987, 8, Supp. 1, S23-S28).

The Use of T Cell Compositions in Angiogenesis

The present invention may also be used to regulate the formation and spreading of blood vessels, or vasculogenesis and angiogenesis, respectively. Both these physiological processes play an important role in wound healing and tissue regeneration.

Initially, at the site of a wound collagen, matrix and blood vessels, are deposited and provide wound strength during tissue repair. The formation of new blood vessels involves the proliferation, migration and infiltration of vascular endothelial cells, and is known to be regulated by a variety of polypeptide growth factors. Several polypeptides with endothelial cell growth promoting activity have been identified, including acidic and basic fibroblastic growth factors (FGF), vascular endothelial growth factor (VEGF), and placental derived growth factor (PDGF).

To stimulate the formation and spreading of blood vessels, activated T cells that express factors that promote the expression of these growth factors, such as, but not limited to, TGF-β, may be administered to the host either into the vasculature or at the site of desired wound healing/angiogenesis. In some instances, it may be necessary to induce the wound healing process through tissue injury.

The activated T cells, or supernatants therefrom, of the present invention may also be used to stimulate angiogenesis in in vitro cultures of cardiomyocytes, endothelial cells or precursors of these cells. Co-culture of these cells could lead to maturation, differentiation, improved function, and/or enhanced engraftment potential of the cells. Further, co-culture of the cells of the present invention with the various cell types in vitro could lead to alteration of function of the cells of non T cell lineage.

Angiogenic agents, including molecules which induce physiological changes in a mammal which are characteristic of angiogenesis modulation, for example, vasoendothelial growth factor, may also be used in conjunction with the compositions of the present invention. Examples of the characteristic modulation include modulation (promotion or suppression) of tumor growth, tissue repair and tissue remodeling. Peptides which modulate tumor growth when incorporated into multivalent ligands are considered to be angiogenic. Also included within the definition of angiogenic agents are molecules which modulate cellular processes involved in the genesis of blood vessels or the expression of endothelial cell phenotypes. Examples include endothelial cell proliferation, endothelial cell survival, endothelial cell motility, binding to endothelial cells.

In vitro assays useful for assessing angiogenesis are described in Tolsma, et al. J. Cell Biol. 122:497 (1993) and Vogel et al. J. Cell. Biochem. 53:74 (1993), hereby incorporated in their entirety by reference. The in vitro assay described in U.S. Pat. No. 6,225,118, hereby incorporated in its entirety, may also be used. Briefly, described therein is an in vitro assay for angiogenesis dependent on appropriate cell signaling mechanisms using a dual culture and requiring no additional growth factors. Both stimulation and inhibition of angiogenesis can be demonstrated using this technique.

The Use of T Cell Compositions in Nerve Regeneration

In another aspect of the present invention, the activated T cells or supernatants therefrom are used to stimulate nerve growth. For this aspect, the compositions described herein can be applied directly to the nerve cells in culture or provided in compositions suitable for in vivo administration. In one preferred aspect of the invention, the compositions are useful for ex vivo nerve regeneration.

According to an alternate embodiment, the method of stimulating neurite outgrowth comprises the additional step of treating a patient or ex vivo nerve cells in culture with a neurotrophic factor. This embodiment includes administering the compositions of the present invention and the neurotrophic agent in a single dosage form or in separate, multiple dosage forms when they are to be administered to a patient. If separate dosage forms are utilized, they may be administered concurrently, consecutively or within less than about 5 hours of one another.

The methods and compositions of this invention may be used to treat nerve damage caused by a wide variety of diseases or physical traumas. These include, but are not limited to, Alzheimer's disease, Parkinson's disease, ALS, multiple sclerosis, stroke and ischemia associated with stroke, neural paropathy, other neural degenerative diseases, motor neuron diseases, sciatic crush, peripheral neuropathy, particularly neuropathy associated with diabetes, spinal cord injuries and facial nerve crush.

Numerous neurotrophic factors have been identified in the art and any of those factors may be utilized in conjunction with the activated T cell or supernatant compositions of this invention. These neurotrophic factors include, but are not limited to, nerve growth factor (NGF), insulin growth factor (IGF-1) and its active truncated derivatives such as gIGF-1, acidic and basic fibroblast growth factor (aFGF and bFGF, respectively), platelet-derived growth factors (PDGF), brain-derived neurotrophic factor (BDNF), ciliary neurotrophic factors (CNTF), glial cell line-derived neurotrophic factor (GDNF), neurotrophin-3 (NT-3) and neurotrophin 4/5 (NT-4/5). One preferred neurotrophic factor in the compositions of this invention is NGF.

The effectiveness of the present invention with respect to nerve regeneration and repair can be measured using the methods described in U.S. Pat. No. 5,547,963, hereby incorporated in its entirety. Briefly, human muscle fragments, cleared of their fibrous sheath, are cut into small pieces and incubated overnight in a conditioning medium consisting of 199 medium with 10% fetal calf serum (FCS) and 1% of a ready-to-use antibiotic and antifungic solution (sodium benzylpenicillinate, streptomycin, fungizone [GIBCO]). These fragments are maintained in a nourishing coagulum consisting of 4 volumes of conditioning medium and 1 volume of human plasma. Explants are then transferred into gelatin-coated Petri dishes, humidified and immobilized on the support by incubation for 1 h at 37° C. and F14 medium (GIBCO), containing 10% FCS, 2 mM glutamine, 10 .mu.g/ml insulin, 10 ng/ml FGF and 10 ng/ml EGF, are added. A large number of satellite muscle cells (precursors of muscular fibers in the adult) migrate outside the explants. These cells start to proliferate and to merge after 1 week in culture. Explants are removed before the satellite cells merge into myotubes. Cells are treated with trypsin just before the merging phase and subculture in order to obtain the amount required for the experiments.

Cells are finally seeded (20,000/cm²). After formation of myotubes, spinal cord explants from 13 day-old rat embryos are immobilized over the muscular cell layer and co-cultured in 25% 199 medium, 67.5% MEM medium (GIBCO), 5% FCS, 10 .mu.g/ml insulin and 1% antibiotic solution. This culture medium is renewed twice a week. Test compounds are dissolved in this culture medium.

Under standard conditions, only 1 out of 4 explants is able to establish functional contacts with muscle fibers. Thus, these experimental conditions are optimal for the demonstration of neuritogenesis and synapse formation.

The effects of the activated T cell or supernatants therefrom of the present invention are determined by measuring the following parameters:
1) Neurite length
2) Neurite length is determined by using a phase-contrast microscope (final magnification 200×) with an ocular micrometer. Neurite length is measured from the center of the explant without taking into account the curving of these filamentous extensions. The length of the branchings is also measured. The total neurite length is determined in at least 15 explants.
3) Number of neurites per explant
4) The number of neurites emerging from each explant is determined without taking into account the branchings.
5) Number of neuromuscular junctions
6) Counting of cholinergic receptor aggregates
7) Cultures are incubated for 1 h in the presence of ¹²⁵I-α-bungarotoxin, fixed with 2.5% glutaraldehyde, dried and dipped in a fluid photographic emulsion. Autoradiograms are developed after 10 days of exposure. Cultures are examined under a microscope (magnification 200×) in order to select isolated muscular fibers with clearly distinct receptor aggregates (these fibers are in general larger than the diameter of the microscope field, and the length of this field is taken as the length unit). At least 60 fibers are studied. Values are the mean of the number of aggregates multiplied by a correction factor and are expressed in mm.
8) Number of acetylcholinesterase-rich synaptic zones
9) Acetylcholinesterase is revealed by the technique of Karnovsky and Roots as modified by Kobayashi and Askanas (J. Neurosci, vol 7, 3131-3141, 1987). Acetylcholinesterase-rich synaptic zones are counted according to the technique described above for receptor aggregates.
10) Surface of the innervated zones
11) Total surface of the innervated muscular cell areas around the explant. This parameter corresponds to the area covered by the motor neurons without taking into account the presence of non innervated zones or other cellular types inside this area.
12) Actual surface covered by innervated muscular fibers. This area is determined by either autoradiographic detection of cholinergic receptor aggregates or by acetylcholinesterase staining and is quantified, after digitalization, by using an image analyzer. This parameter gives an estimation of the number of innervated muscle fibers.
   The Use of T Cell Compositions for Mucositis

Patients undergoing chemotherapy or radiotherapy for treatment of malignancies are almost invariably faced with moderate or severe side effects due to their therapy. One of the common side effects faced by cancer patients is the induction of ulcerative mucositis of the mucosal membranes. This mucositis is especially prominent in the oral cavity. This side effect, although not as life threatening as other side effects such as anemia or immunosuppression, nonetheless often becomes the dose limiting factor in the continuation of therapy in many cancer patients. Ulcerative mucositis is marked by the formation of slowly healing open ulcers in the oral cavity causing a great deal of pain and discomfort to the patient. Eating, drinking, and swallowing become difficult and painful and additionally, the salivary glands are often effected compounding the discomfort. The presence of open ulcers in the mouth often lead to opportunistic infections of bacterial, viral, and fungal origin in these patients, who are often immunologically suppressed due to their therapy. These oral infections must be carefully monitored to avoid their spreading to life-threatening, systemic infections.

As yet, there is no treatment for such mucositis except either cessation of the therapy or palliative and supportive interventions. Some of the palliative treatments in current use include the use of antibiotics to reduce the chance of infection, the use of anti-histamines and anti-inflammatory drugs, and the use of pain reducing medications. All of these treatments are either unacceptable, as with the case of cessation of cancer therapy, or are only partially successful in relieving the suffering from the mucositis.

In one aspect of the present invention, the activated T cells or supernatants therefrom are used in the treatment of mucositis. For this aspect, the compositions described herein can be applied directly to the site of mucositis or provided in pharmaceutical compositions suitable for in vivo administration. In another aspect, the activated T cells or supernatants therefrom may be used to inhibit the development of mucositis. The activated T cells of the present invention may be administered prior to, in conjunction with, or following chemotherapy.

The Use of T Cell Compositions for the Treatment and/or Amelioration of Cachexia

Cachexia involves progressive loss of body weight, anemia, edema and anorexia as cardinal symptoms, which is associated with malignant tumor, tuberculosis, diabetes, homodyscrasia, endocrinopathy, AIDS and so on “J. Parenteral and Enteral Nutrition, 12, 286-298, 1988” and “American Journal of Medicine, 85, 289-291, 1988”. Other clinical manifestations of cachexia may include impaired immune function, early satiety, weakness, poor performance status, tissue and, specifically, muscle wasting and fatigue (1997 Puccio and Nathanson, Seminars in Oncology, 24(3):277-287). Cancer cachexia is one of the worst effects of malignancy and accounts for nearly a third of cancer deaths (1999 Argilés and López-Soriano, Med. Res. Rev, 19(3):223-248). While classically associated with cancer, cachexia is present in patients with a variety of chronic illnesses including autoimmune diseases, HIV, cancer, chronic infections such as hepatitis and tuberculosis, chronic organ failure including renal failure, liver failure, heart failure, chronic obstructive pulmonary disease, and the like. As such, these diseases represent biological states associated with a proinflammatory state and, as discussed further below, can be treated with the T cells of the present invention.

Importantly, many observations implicate cytokines in cachexia (1999 Argilés and López-Soriano, Med. Res. Rev, 19(3):223-248). Among the cytokines that have been implicated in cachexia are TNF-alpha, IL-1, IL-6 and IFN-gamma. All of the illnesses described above are characterized by a decrease in NK and T cells and high levels of pro-inflammatory cytokines including, but not limited to TNF-alpha, IL-1, and IL-6. Many of these illnesses are also characterized by a strong TH2 cytokine profile. IL-12 and IL-2 have been used to stimulate T cells in vivo to reverse cachexia in preclinical and, clinical studies. Unfortunately, these drugs have many side effects. Further, in the presence of other cytokines that counteract their activity, such as in cancer patients, these cytokines may not work optimally. However, treatment with activated T cells, especially using the Xcellerate™ process as described herein, which generates Xcellerated™ T Cells with a strong TH1 cytokine profile may be able to reverse many of these abnormalities by restoring a healthy TH1 versus TH2 cyokine balance as well as suppressing cytokines such as IL-1 and IL-6 that play a major role in cachexia.

Against cachexia, parenteral or enteral nutrition and endocrine therapy, for instance, have been attempted so far but no satisfactory therapeutic modality has been established as yet. Particularly where cachexia is caused by a malignant tumor, progression of cachexia diminishes the tolerance of patients for anticancer chemotherapy so that the treatment encounters a serious setback. On the other hand, palliative nutritional support for cachexia rather may exacerbate the malignant tumor to reduce the survival period of the patient. While cachexia is frequently induced by malignant tumors, administration of antitumor drugs may bring about antitumoral effects but it is the rule rather than exception that side effects of antitumor medication are superimposed to arrest a remission of cachexia. There exists, under the circumstances, a need for a therapeutic drug that would ameliorate or inhibit progression of cachectic symptoms such as loss of body weight.

In one aspect of the present invention, the activated T cells or supernatants therefrom are used in the treatment and/or amelioration of cachexia. For this aspect, the compositions described herein can be provided in compositions (such as pharmaceutical compositions) suitable for in vivo administration. In another aspect, the activated T cells or supernatants therefrom may be used to inhibit the development of cachexia. The activated T cells of the present invention may be administered prior to, in conjunction with, or following chemotherapy. The activated T cells of the present invention may be administered in conjunction with other treatments for cachexia available in the art, including but not limited to, hydrazine sulfate, medroxyprogesterone, megestrol acetate, IL-12, melatonin (M. Puccio and L. Nathanson 1997 Seminars in Oncology, 24:277-287), alpha-lipoic acid, amifostine, N-acetyl cysteine (G. Mantovani, et al., 2003 J Mol Med 81:664-673), thalidomide, pentoxyfyline, eicosapentaenoic acid, and ibuprofen (R. Kurzrock 2001 Cancer 92:1684-1688).

The Use of T Cell Compositions for the Treatment and/or Amelioration of Chronic Diseases

The T cells of the present invention can be used to treat and/or ameliorate chronic diseases. The illustrative example described in further detail herein is chronic renal failure. Chronic renal failure (CRF) occurs as a result of progressive and later, permanent reduction in the glomerular filtration rate (GFR), which is associated with loss of functional nephron units. When the GFR continues to decline to less than 10% of normal (5-10 ml/min), the subject progresses to end-stage renal failure (ESRD). (see for example, R. A. Lafayette, et al., Diseases of the Kidney Eds: R. W. Schrier and C. W. Gottschalk, Little, Brown and Company, Inc., Vol. 6, 307-354 (1997)). Unless the subject receives renal replacement therapy (i.e., chronic hemodialysis, continued peritoneal dialysis or kidney transplantation) renal failure will rapidly progress to cause death.

Chronic inflammation and oxidative stress are common in patients with ESRD. As a result, the main cause of mortality in ESRD is cardiovascular disease. During ESRD, the inability of the remaining nephrons to adequately remove waste products from the blood, while retaining useful products and maintaining fluid and electrolyte balance, leads to a rapid decline in which many organ systems, and particularly the cardiovascular system, may begin to fail. For example, blood urea nitrogen (BUN) and creatinine levels may be expected to rise and, at BUN levels of 60-100 mg/dl and serum creatinine levels of 8-12 mg/dl, a uremic syndrome will typically develop in which the kidneys can no longer remove the end products of nitrogen metabolism. Additionally, several inflammatory markers, such as C-reactive protein, IL-6, fibrinogen, and ICAM-1 are elevated in ESRD patients with clinical signs of cardiovascular disease (P. Stenvinkel and A. Alvestrand 2002 Seminars in Dialysis 15:329-337). Available evidence suggests an up-regulated pro-inflammatory cytokine system activity in ESRD patients and marked elevated levels of cytokines have been found both before and after start of dialysis treatment (see, e.g., J. S. Park and S. B. Kim 2003 Nephrology 8:S40-S44; and P. I. Kimmel et al., 1998 Kidney Int. 54:236-244). Higher levels of circulating proinflammatory cytokines are associated with mortality while improved T cell function is associated with survival in ESRD patients being treated with hemodialysis (P. L. Kimmel, et al., 1998 Supra). Further, patients with chronic renal failure demonstrate impaired immune function characterized by frequent infectious complications, low response to vaccinations and decreased T cell proliferation in response to mitogenic stimuli in vitro due to impaired costimulation by accessory cells (see e.g., H. Kaul, et al., 2000 American Journal of Kidney Diseases, 35:611-616). Thus, treatment with activated T cells, especially using the Xcellerate™ process as described herein, which generates Xcellerated™ T cells with a strong TH1 cytokine profile may be able to reverse many of these abnormalities by restoring a TH1 cyokine balance as well as suppressing inflammatory cytokines such as IL-1 and IL-6 that play a role in chronic renal failure.

Other chronic diseases are also contemplated in the context of the present invention, and include, but are not limited to, heart disease, stroke, diabetes, arthritis, obesity, chronic obstructive pulmonary diseases, Alzheimer's disease, high blood pressure, and the like. Other chronic diseases associated with a proinflammatory state, such as autoimmune diseases can also be treated and/or ameliorated by the T cells of the present invention. Illustrative autoimmune disease include, but are not limited to, rheumatoid arthritis, multiple sclerosis, insulin dependent diabetes, Addison's disease, celiac disease, chronic fatigue syndrome, inflammatory bowel disease, ulcerativecolitis, Crohn's disease, Fibromyalgia, systemic lupus erythematosus, psoriasis, Sjogren's syndrome, hyperthyroidism/Graves disease, hypothyroidism/Hashimoto's disease, Insulin-dependent diabetes (type 1), Myasthenia Gravis, endometriosis, scleroderma, pernicious anemia, Goodpasture syndrome, Wegener's disease, glomerulonephritis, aplastic anemia, any of a variety of cytopenias, paroxysmal nocturnal hemoglobinuria, myelodysplastic syndrome, idiopathic thrombocytopenic purpura, autoimmune hemolytic anemia, Fanconi anemia, Evan's syndrome, Factor VIII inhibitor syndrome, Factor IX inhibitor syndrome, systemic vasculitis, dermatomyositis, polymyositis and rheumatic fever

The T cells of the present invention can be used alone or in conjuction with other treatments known in the art which delay or halt the progression of chronic diseases, such as ESRD. For example, a variety of growth and differentiation factors, e.g., epidermal growth factor (EGF), transforming growth factor-alpha and -beta (TGF-alpha and beta), insulin like growth factor-1 (IGF-1), fibroblast growth factor (FGF), platelet derived growth factor (PDGF) and bone morphogenetic protein (BMP) have been shown to participate in the regulation of the growth and repair of renal tissues. (M. R. Hammerman and S. B. Miller, J. Am. Soc. Nephrolol., 5: 1-11 (1994); and R. C. Harris, Adv. Renal. Repl. Ther., 4: 43-53 (1997)).

The Use of T Cell Compositions for the Treatment and/or Amelioration of Hepatitis and Other Diseases Associated with Chronic Infections

Chronic hepatitis C is an insidious and slowly progressive disease having a significant impact on morbidity and mortality. While many patients who contract hepatitis C will have subclinical or mild disease, HCV infection causes progressive liver damage in the majority of those infected. At least 80% of the individuals who contract HCV will develop chronic infection and hepatitis, a disease state characterized by fluctuating serum transaminase abnormalities and inflammation with or without fibrosis lesions on liver biopsy. Twenty to fifty percent of these will eventually progress to cirrhosis and 1-2% will develop liver cancer after a 10-20 year period.

Similar to cachexia and chronic diseases such as chronic renal failure, elevated serum levels of proinflammatory cytokines (e.g., IL-1, IL-6, tumor necrosis factor alpha) are found in patients with liver disease related to HCV (see, for example, Y.-S. Huang, et al., 1999 Chin Med J 62:327-333). Accordingly, treatment with activated T cells of the present invention may be able to reverse many of these abnormalities by restoring a TH1 cyokine balance as well as suppressing inflammatory cytokines such as IL-1 and IL-6 that play a role in hepatitis.

In this regard, chronic HCV infection is illustrative of other chronic infections. As such, T cells of the present invention can be used in the treatment or amelioration of other chronic infections associated with a proinflammatory state. Thus, the present invention can be used in the treatment and/or amelioration of chronic infections by an y pathogenic agents that may induce a proinflammatory environment, such as viruses (e.g., human immunodeficiency virus), bacteria, parasites and fungi. Thus, any disease that is caused by chronic infection by an infectious organism can be treated and/or ameliorated as described herein. Infectious organisms may comprise viruses, (e.g., single stranded RNA viruses, single stranded DNA viruses, human immunodeficiency virus (HIV), hepatitis A, B, and C virus, herpes simplex virus (HSV), cytomegalovirus (CMV) Epstein-Barr virus (EBV), human papilloma virus (HPV)), parasites (e.g., protozoan and metazoan pathogens such as Plasmodia species, Leishmania species, Schistosoma species, Trypanosoma species), bacteria (e.g., Mycobacteria, in particular, M. tuberculosis, Salmonella, Streptococci, E. coli, Staphylococci), fungi (e.g., Candida species, Aspergillus species), Pneumocystis carinii, and prions (known prions infect animals to cause scrapie, a transmissible, degenerative disease of the nervous system of sheep and goats, as well as bovine spongiform encephalopathy (BSE), or “mad cow disease”, and feline spongiform encephalopathy of cats. Four prion diseases known to affect humans are kuru, Creutzfeldt-Jakob Disease (CJD), Gerstmann-Straussler-Scheinker Disease (GSS), and fatal familial insomnia (FFI)). As used herein “prion” includes all forms of prions causing all or any of these diseases or others in any animals used—and in particular in humans and domesticated farm animals.

Biological parameters relevant to cachexia, chronic diseases such as renal failure, hepatitis, other chronic infections, and other diseases associated with a proinflammatory state that can be measured before and after treatment with the T cells of the present invention include, but are not limited to, serum levels of proinflammatory cytokines (e.g., IL-1 family members such as IL-1α and IL-1β, IL-6, and tumor necrosis factor alpha), acute-phase proteins such as C-reactive protein and fibrinogen, and IL-2/leptin, and those relevant to oxidative stress, such as reactive oxygen species, anti-oxidant enzymes glutathione peroxidase and superoxide dismutase, and other significant clinical indexes of disease progression such as stage, performance status as assessed by the Eastern Cooperative Oncology Group (ECOG-PS) or by the Karnofsky Performance Scale, and body mass index. Further, quality of life measurements can also be taken before and after treatment with the T cells of the present invention, (see for example, Wilson I B, Cleary P D. 1995 JAMA 273(1):59-65.). As would be recognized by the skilled artisan, a reduction in serum levels of any of the proinflammatory cytokines, C-reactive protein, fibrinogen, reactive oxygen species, glutathione peroxidase, or superoxide dismutase is indicative of improvement and can be determined using any of a number assays known in the art (see for example, G. Mantovani, et al., Supra). In certain embodiments, a statistically significant reduction in serum levels of any one or more of the proinflammatory cytokines, C-reactive protein, fibrinogen, reactive oxygen species, glutathione peroxidase, or superoxide dismutase in test subjects following treatment with the T cells of the present invention, as compared to control subjects is desired. Appropriate statistical tests are known to the skilled artisan and numerous statistical analysis programs are commercially available (see for example, Analyse-It Software, Ltd., Leeds, UK; and SPSS Inc., Chicago, Ill.).

The Use of T Cell Compositions in Gene Discovery

The in vitro co-culture of the activated T cells, and/or the supernatants therefrom, described herein may also be applicable to gene discovery. For example, co-culture of the activated T cells and/or supernatants therefrom, with nerve cells, cariomyocytes, endothelial cells, bone cells, and/or precursors of these cells could lead to altered gene expression in the target cells. Cells of interest could then be isolated using various techniques known to skilled artisans such as numerous immunoselection methods. Such techniques are described, for example, in Current Protocols in Immunology, John Wiley & Sons, New York. N.Y. Cells isolated in this manner could then be used in the generation of gene-libraries. DNA or cDNA library construction techniques are well known to those skilled in the art. Custom libraries can also be generated commercially by various companies, such as Clontech (Palo Alto, Calif.). These libraries could then be screened by, for example by PCR and direct sequencing, to identify known and/or unique genes involved in the process of tissue repair and regeneration activated as a result of the activated T cells or supernatants therefrom.

Formulations/Pharmaceutical Compositions

The present invention further provides pharmaceutical compositions comprising the activated T cells, and/or cells altered following co-culture with activated T cells or supernatants therefrom, and a pharmaceutically acceptable carrier. Compositions of the present invention may be administered either alone, or as a pharmaceutical composition in combination with diluents and/or with other components such as IL-2 or other cytokines or cell populations. Briefly, pharmaceutical compositions of the present invention may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as ethylenediaminetetraacetic acid (EDTA) or glutathione; adjuvants (e.g., aluminum hydroxide); and preservatives. Compositions of the present invention are, in certain aspects, formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “a therapeutically effective amount” is indicated, the precise amount of the compositions of the present invention to be administered can be determined by a physician with consideration of individual differences in age, weight, tumor size, extent of infection or metastasis, and condition of the patient. Typically, in adoptive immunotherapy studies, activated T cells are administered approximately at 2×10⁹ to 2×10¹¹ cells to the patient. (See, e.g., U.S. Pat. No. 5,057,423). In some aspects of the present invention, particularly in the use of allogeneic or xenogeneic cells, lower numbers of cells, in the range of 10⁶/kilogram (10⁶-10¹¹ per patient) may be administered. T cell, or other altered post co-culture cell compositions may be administered multiple times at dosages within these ranges. The activated T cells may be autologous or heterologous to the patient undergoing therapy.

The administration of the subject pharmaceutical compositions may be carried out in any convenient manner, including by aerosol inhalation, injection, ingestion, transfusion, implantation or transplantation. The compositions of the present invention may be administered to a patient subcutaneously, intradermally, intramuscularly, by intravenous (i.v.) injection, or intraperitoneally. The T cell compositions of the present invention are preferably administered by i.v. injection. The compositions of activated T cells may be injected directly into a site of tissue injury.

In yet another embodiment, the pharmaceutical composition can be delivered in a controlled release system. In one embodiment, a pump may be used (see Langer, 1990, Science 249:1527-1533; Sefton 1987, CRC Crit. Ref. Biomed. Eng. 14:201; Buchwald et al., 1980; Surgery 88:507; Saudek et al., 1989, N. Engl. J Med. 321:574). In another embodiment, polymeric materials can be used (see Medical Applications of Controlled Release, 1974, Langer and Wise (eds.), CRC Pres., Boca Raton, Fla.; Controlled Drug Bioavailability, Drug Product Design and Performance, 1984, Smolen and Ball (eds.), Wiley, N.Y.; Ranger and Peppas, 1983; J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, thus requiring only a fraction of the systemic dose (see, e.g., Medical Applications of Controlled Release, 1984, Langer and Wise (eds.), CRC Pres., Boca Raton, Fla., vol. 2, pp. 115-138).

The compositions of the present invention may also be administered using any number of matrices. Matrices have been utilized for a number of years within the context of tissue engineering (see, e.g., Principles of Tissue Engineering (Lanza, Langer, and Chick (eds.)), 1997. The present invention utilizes such matrices within the novel context of acting as an artificial lymphoid organ to support, maintain, or modulate the immune system, typically through modulation of T cells. Accordingly, the present invention can utilize those matrix compositions and formulations which have demonstrated utility in tissue engineering. Accordingly, the type of matrix that may be used in the compositions, devices and methods of the invention is virtually limitless and may include both biological and synthetic matrices. In one particular example, the compositions and devices set forth by U.S. Pat. Nos.: 5,980,889; 5,913,998; 5,902,745; 5,843,069; 5,787,900; or 5,626,561 are utilized, as such these patents are incorporated by reference in their entirety. Matrices comprise features commonly associated with being biocompatible when administered to a mammalian host. Matrices may be formed from both natural and synthetic materials. The matrices may be non-biodegradable in instances where it is desirable to leave permanent structures or removable structures in the body of an animal, such as an implant; or biodegradable. The matrices may take the form of sponges, implants, tubes, telfa pads, fibers, hollow fibers, lyophilized components, gels, powders, porous compositions, or nanoparticles. In addition, matrices can be designed to allow for sustained release seeded cells or produced cytokine or other active agent. In certain embodiments, the matrix of the present invention is flexible and elastic, and may be described as a semisolid scaffold that is permeable to substances such as inorganic salts, aqueous fluids and dissolved gaseous agents including oxygen.

A matrix is used herein as an example of a biocompatible substance. However, the current invention is not limited to matrices and thus, wherever the term matrix or matrices appears these terms should be read to include devices and other substances which allow for cellular retention or cellular traversal, are biocompatible, and are capable of allowing traversal of macromolecules either directly through the substance such that the substance itself is a semi-permeable membrane or used in conjunction with a particular semi-permeable substance.

Compositions comprising the activated T cells and/or supernatants therefrom as described herein can be provided as pharmaceutically acceptable formulations using formulation methods known to those of ordinary skill in the art. These formulations can be administered by standard routes. In general, the combinations may be administered by the topical, transdermal, oral, rectal or parenteral (e.g., intravenous, subcutaneous or intramuscular) route. In addition, the combinations may be incorporated into biodegradable polymers allowing for sustained release of the composition, the polymers being implanted in the vicinity of where delivery is desired, for example, at the site of tissue injury. The biodegradable polymers and their use are described, for example, in detail in Brem et al. J. Neurosurg. 74:441-446 (1991).

The dosage of the compositions will depend on the condition being treated, and other clinical factors such as weight and condition of the human or animal, the nature of the composition, and the route of administration of the composition. It is to be understood that the present invention has application for both human and veterinary use.

The formulations include those suitable for oral, rectal, ophthalmic, (including intravitreal or intracameral) nasal, topical (including buccal and sublingual), vaginal or parenteral (including subcutaneous, intramuscular, intravenous, intradermal, intratracheal, and epidural) administration. The formulations may conveniently be presented in a dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into associate the active ingredient with liquid carriers or finely divided solid carriers or both, and then, if necessary, shaping the product.

Formulations suitable for topical administration to the skin may be presented as ointments, creams, gels and pastes comprising the ingredient to be administered in a pharmaceutical acceptable carrier. A preferred topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tamports, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules and tablets of the kind previously described.

Preferred unit dosage formulations are those containing a daily dose or unit, daily sub-dose, as herein above recited, or an appropriate fraction thereof, of the administered ingredient.

It should be, understood that in addition to the ingredients, particularly mentioned above, the formulations of the present invention may include other agents conventional in the art having regard to the type of formulation in question, for example, those suitable for oral administration may include flavoring agents.

In one embodiment of the present invention, compositions comprising cells of the present invention, either activated T cells or cells previously co-cultured with activated T cells are targeted to the desired location through the use of paramagnetic beads and application of a magnetic force inside or outside a target tissue (as described, for example, in U.S. Pat. No. 6,203,487, hereby incorporated by reference in its entirety). Briefly, the cells of the present invention, either activated T cells or cells previously co-cultured with activated T cells, are exposed to paramagnetic beads conjugated to appropriate surface markers either in vivo or in vitro or a combination of the two such that binding of the paramagnetic particle to the cells occurs. If carried out in vitro, a composition comprising cells bound to the paramagnetic particles and a pharmaceutically acceptable excipient is administered to a mammal. A magnet may be placed adjacent to a target tissue, i.e., an area of the body or a selected tissue or organ into which local cell delivery is desired. The magnet can be positioned superficial to the body surface or can be placed internal to the body surface using surgical or percutaneous methods inside or outside the target tissue for local delivery. The magnetic particles bound to cells are delivered either by direct injection into the selected tissue or to a remote site and allowed to passively circulate to the target site or are actively directed to the target site with a magnet or the targeting ligand.

All references referred to within the text are hereby incorporated by reference in their entirety. Moreover, all numerical ranges utilized herein explicitly include all integer values within the range and selection of specific numerical values within the range is contemplated depending on the particular use. Further, the following examples are offered by way of illustration, and not by way of limitation.

### EXAMPLES

### Example 1

### An Animal Modem for Wound Healing

An animal model of superficial (skin) wounds is examined to study the affect of the compositions of the present invention on wound repair and healing.

Split thickness skin wounds, approximately 2×2 cm are made over the back of anesthetized swine according to the method described by Staiano-Coico et al. J Clin Invest. 77(2):396-404-(1986). The pig model is commonly used in such studies as pig skin is most like human skin. A small amount of solution comprising the composition of the present invention is placed onto about 11 wounds and an occlusive adhesive dressing is used to cover the wounds. A placebo solution (saline, 50 to 100 .mu.l) is placed onto each of 11 “mirror”, identical wounds which are also covered with occlusive dressing. After 3 days, the animals are anesthetized, sacrificed and full thickness skin samples twice as large as the original skin wound they contained are removed. The samples are coded to blind the treatment received analyzed by a pathologist and scored for the percent of healing. This scoring method predominantly measures the amount of epithelialization (wound coverage by keratinocytes) that had taken place during the 3 days of repair and healing since the wounding, results for the 11 wounds treated with the compositions of the present invention are compared to the saline controls and expressed as the percent of healing. A higher percent reflects more healing.

### Example 2

### An Animal Model for Myocardial Ischemia

Important prerequisites for successful studies on tissue repair and regeneration using the T cell or supernatants therefrom of the present invention are (a) constitution of an animal model which is applicable to clinical myocardial ischemia which can provide useful data regarding mechanisms for angiogenesis in the setting of myocardial ischemia, and (b) accurate evaluation of the effects of the compositions described herein. A porcine model of myocardial ischemia that mimics clinical coronary artery disease is used, as described in U.S. Pat. No. 6,174,871, hereby incorporated in its entirety. Placement of an ameroid constrictor around the left circumflex (LCx) coronary artery results in gradual complete closure (within 7 days of placement) with minimal infarction (1% of the left ventricle, 4.+−0.1% of the LCx bed) (Roth et al Circulation 82:1778, 1990; Roth et al. Am J Physiol 235:H1279, 1987; White et al. Circ Res 71: 1490, 1992, Hammond et al. Cardiol 23:475, 1994; and Hammond et al. J Clin Invest 92:2644, 1993). Myocardial function and blood flow are normal at rest in the region previously perfused by the occluded artery (referred to as the ischemic region), due to collateral vessel development, but blood flow reserve is insufficient to prevent ischemia when myocardial oxygen demands increase. Thus, the LCx bed is subject to episodic ischemia, analogous to clinical angina pectoris. Collateral vessel development and flow-function relationships are stable within 21 days of ameroid placement, and remain unchanged for four months (Roth et al. Circulation 82:1778, 1990; Roth et al. Am J Physiol 235:H1279, 1987; White et al. Circ. Res 71:1490, 1992). It has been documented by telemetry that animals have period ischemic dysfunction in the bed at risk throughout the day, related to abrupt increases in heart rate during feeding, interruptions by personnel, etc. (unpublished data). Thus, the model has a bed with stable but inadequate collateral vessels, and is subject to periodic ischemia. Another distinct advantage of the model is that there is a normally perfused and functioning region (the LAD bed) adjacent to an abnormally perfused and functioning region (the LCx bed), thereby offering a control bed within each animal.

Myocardial contrast echocardiography is used to estimate regional myocardial perfusion. The contrast material is composed of microaggregates of galactose and increases the echogenicity (whiteness) of the image. The microaggregates distribute into the coronary arteries and myocardial walls in a manner that is proportional to blood flow (Skyba et al. Circulation 90:1513-1521, 1994). It has been shown that peak intensity of contrast is closely correlated with myocardial blood flow as measured by microspheres (Skyba et al. Circulation 90:1513-1521, 1994). To document that the echocardiographic images employed in the present invention are accurately identifying the LCx bed, and that myocardial contrast echocardiography could be used to evaluate myocardial blood flow, a hydraulic cuff occluder was placed around the proximal LCx adjacent to the ameroid.

When animals are sacrificed, the hearts are perfusion-fixed (glutaraldehyde, physiological pressures, in situ) in order to quantitate capillary growth by microscopy. PCR is used to detect angiogenic protein DNA and mRNA in myocardium from animals that had received gene transfer. Finally, using a polyclonal antibody to an angiogenic protein, angiogenic protein expression in cells and myocardium from animals that are administered the compositions of the present invention is examined.

The strategy for therapeutic studies includes the timing of administration of the composition, the route of administration of the compositions, and type of composition (e.g. activated T cells, supernatants therefrom, cells following co-culture with activated T cells). In the ameroid model of myocardial ischemia, the desired composition is performed after stable but insufficient collateral vessels have developed. Those skilled in the art will understand that the results demonstrated in pigs are predictive of results in humans. The pig has a native coronary circulation very similar of that of humans, including the absence of native coronary collateral vessels.

### Example 3

### Repair of Osteoblastic and Lytic Bone Lesions in Two Patients Receiving Activated T Cell (Xcellerate™)+IL-2 Therapy

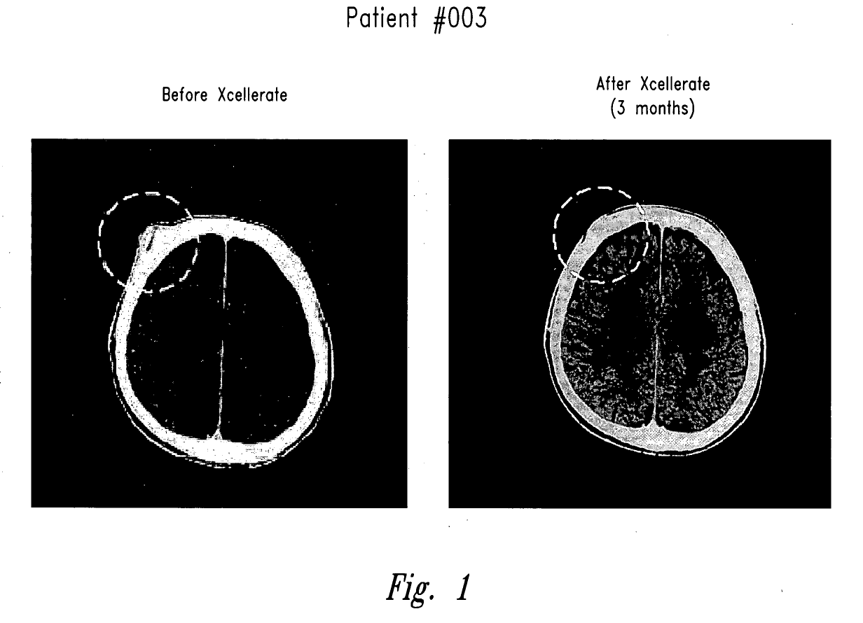

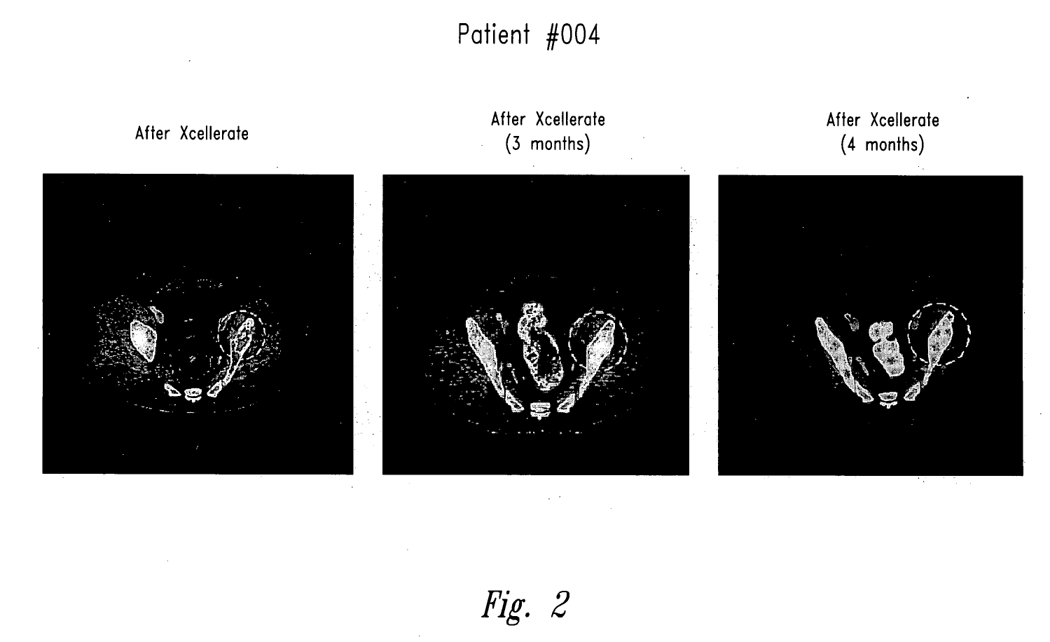

Three patients were entered on a Phase I metastatic renal cell carcinoma trial (XT00) and were receiving treatment with activated T cells (XCELLERATE™)+IL-2 therapy. One patient (Patient #003) had an osteoblastic lesion in his skull that completely resolved after treatment with the XCELLERATE™+IL-2 therapy (FIG. 1, circled region). A second patient (Patient #004) had a large 7 centimeter lytic lesion in his pelvis and a second lytic lesion in his rib. After treatment with XCELLERATE™+IL-2, both bone lesions healed completely and remained healed as of the 10 month follow-up visit (FIG. 2, circled region).

### Example 4

### An in vitro Model for Angiogenesis Using Activated T Cells

An in vitro model of angiogenesis is used, as described in Iruela-Arispe et al. Proc. Nat. Acad. Sci. USA 88:5026-5030, 1991, hereby incorporated in its entirety. Briefly, Bovine aortic and rat vascular endothelial cells (BAEC and RVEC, respectively) are isolated essentially as described in Sage, H. et al. Biochemistry 24:5433-5442, 1979. Clones from BAEC expressing a sprouting phenotype and RVEC clones that organize into endothelial cords, are selected. Both cell types are cultured at 37° C. in Dulbecco's modified Eagle's medium containing 10% (vol/vol) heat inactivated FCS. Cells are used between passages 5 and 10 for BAEC and between 25 and 30 for RVEC. Spontaneous formation of endothelial cords generally occurs 10-15 days after confluence.

BAEC plated on 12-well Costar plates (Corning) from cord within 2 weeks. One cord is defined as the length between two intersecting (vertex) points. To determine the number of tubes, 10 microscope fields (using a ×10 objective and ×1 ocular lenses) are examined in a premarked plate. For each experiment, five replicate cultures are counted (day 0). The cultures are then washed three times with serum-free medium and are incubated with activated T cells or supernatants therefrom. The number of T cells used and the day following activation that the T cells are used is optimized.

From each set of experiments, the mean number of cords±SEM is calculated at day 0 and day 2. Values are also expressed as percentages (±SEM), with the number of cords at day 0 in each culture taken as 100%. The data are analyzed by a paired-sample t test, and the differences are considered significant when P<0.025.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for ameliorating a disease associated with a proinflammatory state in an individual, comprising
a. contacting a population of cells, wherein at least a portion of the population comprises T cells, from an individual afflicted with the disease, with a surface wherein said surface has attached thereto a first agent which stimulates a TCR/CD3 complex-associated signal in the T cells and a second agent that binds the CD28 accessory molecule on the surface of the T cells, thereby activating the T cells;
b. administering the activated T cells to the individual;
thereby ameliorating the disease associated with a proinflammatory state in the individual.

2. The method of claim 1 wherein the disease is a chronic inflammatory condition selected from the group consisting of chronic cardiac disease, chronic lung disease, chronic renal failure, hepatitis, chronic autoimmune disease, and chronic infections.

3. The method of claim 1 wherein the disease is cachexia.

4. The method of any one of claims 1-3 wherein the ameliorating comprises a reduction in serum levels of one or more proinflammatory cytokines as compared to levels prior to administering the activated T cells.

5. The method of claim 3 wherein the treatment leads to an increase in body weight.

6. The method of claim 3 wherein the treatment leads to any one or more of an increase in energy level, an increase in ECOG performance status, and an increase in Karofsky performance status.

7. The method of claim 1 wherein the first agent is an antibody or an antigen-binding fragment thereof.

8. The method of claim 7 wherein the antibody or antigen-binding fragment thereof is a monoclonal antibody or antigen-binding fragment thereof.

9. The method of claim 7 wherein the antibody is an anti-CD3 antibody.

10. The method of claim 1 wherein the second agent is an antibody or an antigen-binding fragment thereof.

11. The method of claim 10 wherein the antibody or antigen-binding fragment thereof is a monoclonal antibody or antigen-binding fragment thereof.

12. The method of claim 10 wherein the antibody is an anti-CD28 antibody.

13. The method of claim 1 wherein the first and the second agents are both antibodies or antigen-binding fragments thereof.

14. The method of claim 13 wherein the first agent is an anti-CD3 antibody or antigen-binding fragments thereof and the second agent is an anti-CD28 antibody or antigen-binding fragments thereof.

15. The method of claim 1 wherein the second agent is a natural ligand of CD28.

16. The method of claim 15 wherein the natural ligand is B7-1.

17. The method of claim 1 wherein said surface is a solid surface.

18. The method of claim 1 wherein said surface is a cell surface.

19. The method of claim 1 wherein said surface is a paramagnetic bead.

20. The method of claim 1 wherein said first and said second agent are covalently attached to said surface.

21. The method of claim 1 wherein said first and said second agent are noncovalently attached to said surface.

22. The method of claim 1 wherein said first and said second agent are indirectly attached to said surface.

23. A method for treating a disease associated with a proinflammatory state, comprising administering activated T cells to an individual afflicted with the disease;
thereby treating the disease associated with a proinflammatory state.

24. The method of claim 23 wherein the disease is a chronic inflammatory condition selected from the group consisting of chronic cardiac disease, chronic lung disease, chronic renal failure, hepatitis, chronic autoimmune disease, and chronic infections.

25. The method of claim 23 wherein the disease is cachexia.

26. The method of any one of claims 23-25 wherein the treatment results in a reduction in serum levels of one or more proinflammatory cytokines as compared to levels prior to administering the activated T cells.

27. The method of claim 23 wherein the treatment leads to an increase in body weight.

28. The method of claim 23 wherein the treatment leads to any one or more of an increase in energy level, an increase in ECOG performance status, and an increase in Karofsky performance status.
